(19) United States
(12) Patent Application Publication
(10) Pub. No.: US 2020/0308614 A1
(43) Pub. Date: Oct. 1, 2020

(54) OPTIMIZED METHOD FOR DECONTAMINATING PRODUCTION OF GLUCOSE POLYMERS AND GLUCOSE POLYMER HYDROLYZATES

(71) Applicant: Roquette Freres, Lestrem (FR)

(72) Inventors: Pierre LANOS, La Bassee (FR); Sophie DUVET, Quesnoy Sur Deule (FR); Thierry DUPONT, Vendin Les Bethune (FR); Fabrice ALLAIN, Lille (FR); Mathieu CARPENTIER, Saint Andre Lez Lille (FR); Agnès DENYS, Lille (FR); Hèla HACINE-GHERBI, Villeneuve D'ascq (FR)

(21) Appl. No.: 16/847,716

(22) Filed: Apr. 14, 2020

Related U.S. Application Data
(63) Continuation of application No. 15/126,293, filed on Sep. 15, 2016, now abandoned.

(30) Foreign Application Priority Data
Mar. 21, 2014 (FR) 14 52354

(51) Int. Cl. C12P 19/14; C08B 37/00; G01N 33/68

(52) U.S. Cl. CPC C12P 19/14; C08B 37/0009; C12Y 302/01078; G01N 33/6863; C08B 37/0024

(57) ABSTRACT

The present invention relates to a method for decontaminating glucose polymers or the hydrolysates of the pro-inflammatory molecules thereof. Said method includes a) providing glucose polymers or the hydrolysates thereof, b) optionally, detecting or assaying the pro-inflammatory molecules in the glucose polymers or the hydrolysates thereof provided in Step a), and c) carrying out the following purifying steps: i. treatment using an enzymatic preparation having detergent properties and clarification properties; ii. treatment using a pharmaceutical-grade activated carbon with very high adsorption properties and “micropore” porosity; iii. optionally, treatment using a second activated carbon with “mesopore” porosity; iv. passing them over a macroporous adsorbent polymer resin having porosity greater than 100 Angstroms; and v. continuous ultrafiltration at 5 kDa.

The present invention relates to the development of an optimized method for decontaminating circuits for producing or purifying glucose polymers, more particularly those intended for the food (fiber-rich health ingredients) and medical (peritoneal dialysis) sectors, or glucose polymer hydrolyzates, more particularly those intended for the medical sectors (apyrogenic injectable glucose).

### TECHNOLOGICAL BACKGROUND OF THE INVENTION

The Applicant company has chosen to develop its invention in a field which is known for the dangerousness of the contaminants of microbial origin capable of developing in circuits for producing glucose polymers or in those for producing hydrolyzates thereof, said contaminants being the source of possible:
food poisoning,
inflammatory reactions which are very harmful to human health.

In the context of a food safety approach, as in that of a health safety approach, it is therefore important to be sure of the absence of contaminants of microbial origin, both in the form of living cells and in the form of cell debris, by all appropriate technical means, especially:
the definition of methods for effective identification and assaying of contaminants,
the definition of safe production circuits, by setting up appropriate purification devices and techniques.

In the case of peritoneal dialysis, a certain number of ingredients must be prepared under the strictest conditions of purity.

Peritoneal dialysis is in fact a type of dialysis, the aim of which is to remove waste such as urea, creatinine, excess potassium or surplus water that the kidneys cannot manage or can no longer manage to purify out of the blood plasma. This medical treatment is indicated in the event of end-stage chronic renal failure.

The dialyzates most commonly used are composed of a buffer solution (of lactate or of bicarbonate) at acidic pH (5.2-5.5) or physiological pH (7.4) to which are added:
electrolytes (sodium, calcium, magnesium, chlorine) and above all
an osmotic agent, principally a glucose polymer, such as for example “icodextrin” present in the ambulatory peritoneal dialysis solution EXTRANEAL® sold by BAXTER.

In this more particular field of the use of glucose polymers intended for continuous ambulatory peritoneal dialysis, it very quickly became apparent that these starch hydrolyzates (mixture of glucose, and of glucose oligomers and polymers) could not be used as such.

European patent application EP 207 676 teaches that glucose polymers forming clear and colorless solutions at 10% in water, having a weight-average molecular weight (Mw) of 5 000 to 100 000 daltons and a number-average molecular weight (Mn) of less than 8 000 daltons, are preferred.

Such glucose polymers also preferably comprise at least 80% of glucose polymers of which the molecular weight is between 5 000 and 50 000 daltons, little or no glucose or glucose polymers with a DP less than or equal to 3 (molecular weight 504) and little or no glucose polymers with a molecular weight greater than 100 000 (DP of about 600).

In other words, the preferred glucose polymers are glucose polymers with a low polydispersity index (value obtained by calculating the Mw/Mn ratio).

The methods proposed in that patent application EP 207 676 for obtaining these glucose polymers with a low polydispersity index from starch hydrolyzates consist:
either in carrying out a fractional precipitation of a maltodextrin with a water-miscible solvent,
or in carrying out a molecular filtration of this same maltodextrin through various membranes possessing an appropriate cut-off or exclusion threshold.

In the two cases, these methods are aimed at removing both the very high-molecular-weight polymers and the low-molecular-weight monomers or oligomers.

However, these methods are not satisfactory both from the point of view of their implementation and from the point of view of the yields and the quality of the products that they make it possible to obtain.

In the interests of developing a method for producing a completely water-soluble glucose polymer with a low polydispersity index preferentially less than 2.5, preferably having an Mn of less than 8 000 daltons and having an Mw of between 12 000 and 20 000 daltons, said method lacking the drawbacks of the prior art, the Applicant company endeavored to solve this problem in its patent EP 667 356, by starting from a hydrolyzed starch rather than from a maltodextrin.

The glucose polymer obtained by chromatographic fractionation then preferably contains less than 3% of glucose and of glucose polymers having a DP less than or equal to 3 and less than 0.5% of glucose polymers having a DP greater than 600.

It is finally henceforth accepted by experts in the field of peritoneal dialysis that these glucose polymers, used for their osmotic power, are entirely satisfactory.

Risks of Contamination

However, risks of microbial contamination of the preparations intended for peritoneal dialysis are to be deplored.

Indeed, it is known that glucose polymer production circuits can be contaminated with microorganisms, or with pro-inflammatory substances contained in said microorganisms.

The contamination of corn or wheat starches with microorganisms of yeast, mold and bacteria type, and more particularly with acidothermophilic bacteria of Alicyclobacillus acidocaldarius type (extremophilic bacteria which grow in the hot and acidic zones of the circuit) is, for example, described in the starch industry.

The major risk for the patient who receives these contaminated products is then peritonitis.

These episodes of peritonitis are caused by intraperitoneal bacterial infections, and the diagnosis is usually easily established through positive dialyzate cultures.

“Sterile peritonitis”, which is described as aseptic, chemical or culture-negative peritonitis, is, for its part, typically caused by a chemical irritant or a foreign body.

Since the introduction of icodextrin for the preparation of peritoneal dialysis solutions, isolated cases of aseptic peritonitis have been reported that can be linked to various causes, especially induction by pro-inflammatory substances potentially present.

Aseptic inflammatory episodes are therefore major complications observed after injections of dialysis solutions.

While some of these inflammatory episodes are linked to a problem of chemical nature (accidental injection of chemical contaminants or incorrect doses of certain compounds), the majority of cases are directly associated with the presence of contaminants of microbial origin that are present in the solutions used to prepare the dialysis solutions.

Lipopolysaccharides (LPSs) and peptidoglycans (PGNs) are the main contaminants of microbial origin with a high risk of triggering an inflammation, even when they are present in trace amounts.

It is, moreover, to the Applicant company's credit to have also taken into account the presence of molecules capable of exacerbating the inflammatory response induced by these contaminants, such as PGN depolymerization products, the minimum structure of which that is still bioactive being muramyl dipeptide (MDP).

In addition to the PGN depolymerization products, formylated microbial peptides, the prototype of which is f-MLP (formyl-Met-Leu-Phe tripeptide), also have a substantial synergistic activity. Originally, these peptides were identified for their chemoattractant activity on leukocytes, although they are incapable of inducing a cytokine response per se.

It is therefore important not to overlook these “small molecules”, since they can indirectly account for aseptic inflammatory episodes by exacerbating the effects of traces of PGN and/or of LPS.

Definition of Methods for Effective Identification and Assaying of Said Contaminants

The Applicant company has therefore devoted itself to developing detection and assaying methods which are more effective than those accessible in the prior art.

Over the last few years, many tests using primary cells have been developed in order to replace animal models in inflammatory response tests.

However, these in vitro models are subject to considerable interindividual variability, which can be responsible for experimental biases.

Conversely, monocyte cell lines give consistent responses, thereby explaining why the tests currently being developed increasingly use cells of this type in culture. However, these tests have the drawback of giving an overall inflammatory response to all the contaminants present as a mixture in a solution, and consequently do not make it possible to characterize the nature of the contaminant.

It is also important to note that the exacerbated inflammatory response is visible for cytokines of the acute phase of the inflammation, such as:
TNF-α (Tumor Necrosis Factor alpha),
IL-1β (interleukin 1β) and
chemokines such as CCL5 (Chemokine (C-C unit) ligand 5)/RANTES (Regulated upon Activation, Normal T-cell Expressed, and Secreted), but is not, or barely, visible for IL-6 (interleukin 6).

Thus, the methods based on the production of IL-6 (US 2009/0239819 and US 2007/0184496) are not suitable for detecting contaminants as a mixture in a solution.

It was therefore to the Applicant company's credit to have developed, in its international patent application WO 2012/143647, sensitive and effective methods for detecting microbial contaminants which have a pro-inflammatory action, below the threshold of sensitivity of the procedures currently used and/or described in the literature, and subsequently to have identified the family, or even the nature, of the pro-inflammatory molecules present in trace amounts in the batches originating from the production circuits.

Determining the Effectiveness of the Individual Purification Steps

The Applicant company then sought to better define the key purification steps to be carried out to ensure optimum safety for the production lines, especially those for glucose polymers.

To this end, it devoted itself to validating the key individual steps for purification of said circuits, by using the detection and assaying methods based on monocyte lines as presented in its intentional patent application WO 2012/143647.

Thus, in its international patent application WO 2013/178931, the Applicant company analyzed the effectiveness of the following individual steps:
heat treatment,
acidification,
passing over activated carbon,
passing over adsorption, ultrafiltration or filtration resins,
chemical or enzymatic hydrolysis.

To analyze the effectiveness of these different individual steps, various decontamination steps were then carried out on various matrices:
glucose polymers, raw materials of icodextrin (before chromatographic fractionation according to the teaching of patent EP 667 356),
a batch of icodextrin,
a batch of branched maltodextrin, sold by the Applicant company under the brand name NUTRIOSE® FB06,
a batch of dextrose monohydrate prepared so as to be conditioned in an injectable solution, sold by the Applicant company under the brand name LYCADEX® PF,
a batch of highly-branched soluble glucose polymers, prepared according to the teaching of international patent application WO 2007/099212 of which the Applicant company is the proprietor,
a commercial maltodextrin.

While these various studies have made it possible to demonstrate the repercussions that each of these steps could have on the elimination of given contaminants for each of the matrices, it was still necessary to define the best combination able to secure all matrices from all potential contaminants.

From all the results presented in patent application WO 2013/178931, there therefore remained an unsatisfied requirement to develop an optimized method for decontaminating circuits for producing or purifying glucose polymers, more particularly those intended for the food (fiber-rich health ingredients) and medical (peritoneal dialysis) sectors, or glucose polymer hydrolyzates, more particularly those intended for the medical sectors (apyrogenic injectable glucose).

### SUMMARY OF THE INVENTION

The present invention therefore proposes a combination of several decontamination steps carefully selected and placed in order, which proves effective in eliminating all the inflammatory molecules that may be present in the production circuits, especially glucose polymers, irrespective of the nature of the contamination.

The method of the invention thus relates to the following combination of steps:
treatment by an enzymatic preparation with detergent and clarifying properties,
treatment by a pharmaceutical-grade activated carbon with very high adsorption capacity and “microporous” porosity,
optionally, treatment by a second activated carbon with “mesoporous” porosity,
passing over a macroporous adsorbent polymer resin having porosity of greater than 100 angstrom, and
continuous 5 kDa ultrafiltration.

Within the context of the invention:
“enzymatic preparation with detergent and clarifying properties” is intended to mean enzyme activity of mannanase type, such as Mannaway® sold by Novozymes;
“pharmaceutical-grade activated carbon with very high adsorption capacity and “microporous” porosity is intended to mean an activated carbon with porosity equivalent to Norit C Extra USP activated carbon;
“activated carbon with “mesoporous” porosity is intended to mean an activated carbon with porosity equivalent to ENO-PC activated carbon;
“macroporous adsorbent polymer resin having porosity of greater than 100 angstrom” is intended to mean a resin of DOWEX OPTIDORE SD2 type.

Preferably, the method comprises the 5 steps.

In particular, the glucose polymers are selected from icodextrin and maltodextrins, in particular branched or unbranched maltodextrins, and the glucose polymer hydrolyzates are a product of total hydrolysis, such as dextrose monohydrate.

The steps selected make it possible to target the various families of contaminants and thus to obtain products devoid of inflammatory reactivity.

### DETAILED DESCRIPTION OF THE INVENTION

The method of the invention is intended to replace the routes conventionally used to purify glucose polymers or the hydrolyzates thereof.

The method for decontaminating glucose polymers or the hydrolyzates thereof in accordance with the invention comprises the following steps:
a) providing glucose polymers or hydrolyzates thereof;
b) optionally, detecting or assaying the pro-inflammatory molecules in the glucose polymers or hydrolyzates thereof provided in step a);
c) carrying out the following purification steps:
   i. treatment by an enzymatic preparation with detergent and clarifying properties,
   ii. treatment by a pharmaceutical-grade activated carbon with very high adsorption capacity and “microporous” porosity,
   iii. optionally, treatment by a second activated carbon with “mesoporous” porosity,
   iv. passing over a macroporous adsorbent polymer resin having porosity of greater than 100 angstrom, and
   v. continuous 5 kDa ultrafiltration.

The glucose polymers or the hydrolyzates thereof may be intended for peritoneal dialysis, enteral and parenteral feeding and the feeding of neonates.

In one preferred embodiment, the glucose polymers which will be prepared within the context of the present invention are icodextrin or maltodextrins (which are branched or unbranched, as will be described below).

The glucose polymer hydrolyzates concerned here are understood to be, especially, the product of total hydrolysis, such as apyrogenic dextrose monohydrate, sold under the trade name LYCADEX® PF by the Applicant company.

They may be decontaminated at one or several stages of their preparation, and especially at the latter steps of their preparation method.

Thus, the glucose polymers or the hydrolyzates thereof provided in the methods according to the present invention correspond to the product preceding the final product.

The pro-inflammatory components are above all molecules of bacterial origin.

They may be, in particular:
PGNs,
LPSs,
lipopeptides.
PGN depolymerization products, especially MDP,
formylated microbial peptides, such as f-MLP,
β-glucans,
etc.

The methods for measuring the in vitro inflammatory responses which are used in the context of the present invention to monitor the effectiveness of the decontamination steps of the methods for preparing glucose polymers for therapeutic use in humans (e.g. peritoneal dialysis solutions) are based on cell tests (“bio-assays”) using lines of monocyte/macrophage type (THP-1, and/or Raw-Blue™) and transfected lines expressing a specific natural immunity receptor (HEK-Blue™), which cell tests were developed by the Applicant company and detailed in its prior patent applications.

Five lines are preferably used:
Raw-Blue™ line: this line, derived from mouse macrophages, responds to the majority of the pro-inflammatory contaminants that may be present in the glucose polymer matrices and derivatives (PGN, lipopeptides, LPS, zymosan, LTA). Its use therefore makes it possible to estimate the overall load of pro-inflammatory molecules present in the samples.
HEK-Blue™ hTLR2 line: this line, expressing the hTLR2 receptor, specifically responds to TLR2 agonists (PGN and lipopeptides above all). Its use therefore makes it possible to determine the level of these contaminants in the triggering of inflammatory responses.
HEK-Blue™ hTLR4 line: this line, expressing the hTLR4 receptor, specifically responds to LPSs. Its use therefore makes it possible to determine the level of these contaminants in the triggering of inflammatory responses.
HEK-Blue™ hNOD2 line: this line, expressing the hNOD2 receptor, specifically responds to NOD2 agonists. Its use therefore makes it possible to determine the level of MDP and related molecules in the triggering of inflammatory responses.
HEK-Blue™ Null2 line: this is a control line which has not been transfected with an immunity receptor. Its use is necessary to verify that the solutions of glucose polymers or of hydrolyzates thereof do not induce SEAP production via a toxicity mechanism.

However, it should be noted that those skilled in the art may also use other commercial lines (IMGENEX) or they may prepare them.

In one preferred embodiment, the cell lines are used at a density between 0.5 and 1×10⁶ cells/ml of culture medium, and the bringing of the preparation of glucose polymers or hydrolyzates thereof into contact with the cells lasts approximately 16 to 24 h.

Quantification of the contaminants may be carried out using a dose-response curve. This dose-response curve may especially be produced with the same cells, under the same conditions, with increasing doses of contaminants. The dose-response curves are in particular produced with LPS, PGN, lipopeptide and MDP standards.

Preferably, such a dose-response curve can be produced for cells expressing TLR4 (for example, THP-1, HEK-Blue™ hTLR4 and Raw-Blue™) with increasing doses of LPS, for cells expressing TLR2 (for example, THP-1, HEK-Blue™ hTLR2 and Raw-Blue™) with increasing doses of PGN, and for cells that are reactive via NOD2 (for example, HEK-Blue™ hNOD2) with increasing doses of MDP.

The cell tests may be carried out as described in the Applicant's patent applications: WO2012/143647 and WO2013/178931.

The first decontamination step of the method in accordance with the invention consists of a treatment by an enzymatic preparation with detergent and clarifying properties.

This enzyme activity of mannanase type, such as the enzymatic preparation Mannaway® sold by Novozymes, has proven effective for dissociating macrocomplexes such as bacterial debris and high molecular weight PGNs.

Its activity is optimal when it is used at a final concentration of 0.4% (vol/vol) in the 32% (weight/vol) glucose polymer solution, adjusted to pH 10 with NaOH. for a treatment time of 24 h at 50° C.

After treatment, the solution is neutralized by HCl and the enzyme is inactivated by heating at 85° C. for 10 mins.

The second step consists of a treatment by a pharmaceutical-grade activated carbon with very high adsorption capacity and “microporous” porosity.

The Applicant company recommends using an activated carbon of Norit C Extra USP type. This is because C Extra USP carbon proves effective in eliminating PGNs and their degradation products.

Its activity is optimal when it is added at the final concentration of 0.5% (weight/volume) in the 32% (weight/vol) glucose polymer solution, adjusted to pH 4.5 with HCl. The treatment is carried out under stirring for 1 h at 80° C. After treatment, the solution is neutralized by NaOH then filtered through 0.22 μm.

The third step consists of a treatment by a second activated carbon with “mesoporous” porosity. This step is optional.

Here, an activated carbon of ENO-PC type is preferred. This quality of activated carbon has a broad spectrum of action and makes it possible preferentially to eliminate molecules with a molecular weight of <100 kDa (for example, LPS and degradation products of PGNs).

It is also used here at a content of 0.5% at pH 4.5 for 1 h at a temperature of 80° C.

The fourth step consists of a treatment on a macroporous adsorbent polymer resin having a porosity of greater than 100 angstrom.

Dowex SD2 resin is chosen, which has a broader spectrum of elimination of contaminating molecules (other than PGNs) than other resins of the same family.

The 32% (250 ml) glucose polymer solutions are eluted on a column containing 20 ml of this resin.

The final step consists of filtration on an ultrafiltration membrane having a cut-off threshold of 5 kDa.

The aim of the treatment by ultrafiltration is to eliminate the molecules of small size that are still present in the glucose polymer solutions. The Applicant company recommends using this treatment at the end of the method, since this treatment also has a dialysis effect which makes it possible to eliminate traces of salts which have accumulated over the course of the preceding treatments.

It is chosen to continuously inject the glucose polymer solution over a 5 kDa filter at a rate of 25 ml/min for 3 h at room temperature. To compensate for the loss of filtrate, the retentate is injected into the starting solution and continually adjusted to the initial volume (100 ml) by addition of sterile PBS buffer. After 3 h, the volume of filtrate is between 150 and 200 ml, which is greater than the initial volume of the glucose polymer solution.

As will be shown in the examples below, this combination of steps alone is able to provide maximum protection for the circuits for producing polymers and glucose and derivatives thereof from contaminants of bacterial origin.

The invention will be understood more clearly from the following examples which are intended to be illustrative and nonlimiting.

### EXAMPLES

### Example 1: Establishment of the Dose-Response Curves

The dose-response curves are produced with standard agonist molecules: LPS, PGN, PAM3(cys) (PAM₃Cys-Ser-(Lys)4 trihydrochloride, a synthetic lipopeptide), LTA, zymosan and MDP. The Raw-Blue™ and HEK-Blue™ hTLR2, hTLR4, hNOD2 and Null lines are incubated with increasing concentrations of agonists, and the cell response is measured by quantifying the SEAP activity. TNF-α is used as positive control for cell activation:
Raw-Blue™ line (FIG. 1): the cells respond to the major inflammatory molecules that may be present in the matrices and in the glucose polymer derivatives (PGN, lipopeptides, LPS, zymosan, LTA); they especially have a strong reactivity with respect to PGNs, but do not respond to its depolymerization products,
HEK-Blue™ hTLR2 line (FIG. 2): strong reactivity with respect to PGNs and the PAM3(cys) lipopeptide; the cells respond more weakly to the other TLR2 ligands (LTA, zymosan) and show no reactivity with respect to LPSs and to MDP,
HEK-Blue™ hTLR4 line (FIG. 3): strong reactivity with respect to LPSs; the cells respond very weakly to zymosan and show no reactivity with respect to PGNs, lipopeptides, LTA and MDP,
HEK-Blue™ hNOD2 line (FIG. 4): strong reactivity with respect to MDP,
HEK-Blue™ Null2 line (FIG. 5): control for absence of cell toxicity.

### Example 2: Preparation of the Various Glucose Polymer Matrices

As indicated above, the matrices are as follows:
4 glucose polymers, raw materials of icodextrin (before chromatographic fractionation according to the teaching of patent EP 667 356), referenced here E1565, E3063, E1242 and E5248

The preparation of these polymers is carried out in accordance with the teachings of patent application WO 2012/059685;
a contaminated batch of icodextrin (referenced here E209J) and a “negative control” batch of icodextrin, i.e. control for non-contamination in the cell tests (referenced here P11-11). These batches are prepared according to the teaching of patent EP 667 356, described in detail in example 1 of patent application WO 2010/125315.

### Example 3: Analysis of the Cell Responses Induced by the Untreated Samples

The aim of these tests is to determine the pro-inflammatory reactivity and the nature of the contaminants present in the various matrices.

The samples according to example 2 are prepared at 32% (weight/volume) in non-pyrogenic water (for injection).

The assays of the LPS and PGN levels were carried out prior to the cell tests using the SLP-HS and LAL assays (data presented below):

| | P11-11 | E1242 | E1565 | E3063 | E5248 | E209J |
|---|---|---|---|---|---|---|
| SLP-HS PGN | <3 | 21 | 2320 | 16185 | 116 | 393 |
| (ng/g) | | | | | | |
| LAL LPS (EU/g) | <0.3 | 2.4 | 38.4 | 2.4 | 9.6 | 0.6 |
| Modified LAL | <0.3 | 1.2 | 4.8 | 1.2 | 0.3 | <0.3 |
| LPS (EU/g) | | | | | | |

The presence of biocontaminants in the various matrices was analyzed by means of the five cell types, so as to have an overview of the inflammatory responses to certain contaminants (FIG. 6).

For the cell tests, the samples are diluted to 1/10 in the cell culture medium (final concentration: 3.2% (w/v)).

The analyses are carried out on:
Raw-Blue™ line: any contaminants with high reactivity for PGNs,
HEK-Blue™ hTLR2 line: high reactivity for PGNs and lipopeptides,
HEK-Blue™ hTLR4 line: high reactivity for LPSs,
HEK-Blue™ hNOD2 line: MDP and PGN depolymerization products,
HEK-Blue™ Null2 line: control for absence of cell toxicity.

Assessment by Sample:
E1242: high-intensity inflammatory response in the HEK-TLR2 cells, and low-intensity inflammatory response in the HEK-NOD2 cells, evidence of a strong contamination with barely degraded PGNs.
E1565: high-intensity inflammatory response in the HEK-TLR2 cells, medium-intensity inflammatory response in the HEK-TLR4 cells and very high-intensity inflammatory response in the HEK-NOD2 cells, evidence of a strong contamination with degraded PGNs and with LPSs.
E3063: saturated inflammatory response in the HEK-TLR2 cells, medium-intensity inflammatory response in the HEK-TLR4 cells and low-intensity inflammatory response in the HEK-NOD2 cells, evidence of a very strong contamination with barely degraded PGNs and with traces of LPSs.
E5248: low-intensity inflammatory response in the various lines, evidence of a low contamination with PGNs and with LPSs.
E209J: high-intensity inflammatory response in the HEK-TLR2 cells, and low-intensity inflammatory response in the HEK-NOD2 cells, evidence of a strong contamination with weakly degraded PGNs.

None of the glucose polymer matrices gives a response in the presence of HEK-Null cells, which confirms the absence of cytotoxicity.

### Example 4: Analysis of the Effect of the Decontamination Procedures on the Cell Responses Induced by the Samples

In its international patent application WO 2013/178931, the Applicant company enabled identification of the treatments best suited to each type of contaminants present in the samples, and made it possible to determine the experimental conditions for their application to glucose polymer matrices.

On the basis of this earlier study, the numerous individual treatments proposed are:
treatment over activated carbon,
ultrafiltration through a membrane with a 5 kDa filtration threshold,
passage over adsorption resins,
treatment by enzymatic preparations.

In the experiments with the combinations described below, the choice and the arrangement of the steps was determined as a function of:
the experimental treatment conditions,
the levels of contamination in the samples, and
the nature of the pro-inflammatory molecules.

Use of Mannaway®

The teachings of international patent application WO 2013/178931 showed that the enzymatic preparation is slightly contaminated by traces of LPS. In addition, traces of enzyme may persist after treatment in the glucose polymer solution. So as to eliminate these exogenous contaminations, the Applicant company therefore recommends placing the enzymatic treatment step at the start of the procedure in the tests of combinations.

Use of Activated Carbons

The activated carbons retained for the present study are:
C EXTRA USP, for its effectiveness in eliminating PGNs and their degradation products;
ENO-PC, for its preferential action on contaminants of molecular weight <100 kDa (for example, LPS and degradation products of PGNs);
A SUPRA EUR, for its effectiveness in eliminating complexes of high molecular weight.

The Applicant company has chosen to use the activated carbons alone or combined in pairs in the various combinations, and given that the treatments by carbons are carried out batchwise and require heating, neutralization and filtration steps, they are carried out just after the enzymatic treatment but before the other treatments.

Use of Resins

The resins retained are:
Macronet MN-150, for its effectiveness in retaining molecules of low molecular weight (for example MDP and degradation products of PGNs);
Dowex SD2, for its broad spectrum of elimination of contaminants, except PGNS.

Use of Membrane Separation

The aim of the treatment by 5 kDa ultrafiltration is to eliminate the molecules of small size that are still present in the glucose polymer solutions. In addition, this procedure has a dialysis effect and makes it possible to eliminate traces of salt that have accumulated over the course of the previous treatments.

This step is therefore systematically placed at the end of the procedure.

After each step, samples are taken under sterile conditions and are used in the cell tests, so as to assay the overall inflammatory load (Raw-Blue™ cell response) and the amounts of biocontaminants (HEK-Blue™ responses). The cell responses obtained after each step are compared to the response induced by the starting matrix, so as to estimate the effectiveness of the decontamination procedures. The results are expressed as activity relative to the maximum cell response. In all the tests, a non-contamination control is carried out with a solution of P11-11 icodextrin.

### Example 5: Comparative Analysis of Various Combinations of Individual Treatment Steps

A large number of possible combinations may be implicitly deduced from the teaching of patent application WO 2013/178931.

The results obtained for 3 of the conceivable procedures are as follows.

Procedure 1: E1242 Matrix; Treatment by C EXTRA USP+ENO-PC Activated Carbons; Passage Over Macronet MN-15 Resin; 5 kDa Ultrafiltration

The results of the cell tests are given in FIG. 7.

The E1242 matrix induces an intermediate inflammatory response in the Raw-Blue cells, which is predominantly linked to a high concentration of PGN (TLR2 response).

It also contains traces of LPS and of degradation products of PGNs, given that the TLR4 and NOD2 responses are close to background noise.

The decontamination procedure effectively reduces the inflammatory responses.

Nonetheless, the response of the Raw-Blue cells remains slightly higher than the non-contamination control (P11-11), indicating that traces of contaminants are still present.

This result is due to the presence of residual PGNs after decontamination. Indeed, the TLR2 response is greatly reduced after treatment by the carbons, but remains higher than the negative control.

The other steps do not have an effect on the TLR2 response, which does not evolve any more up to the end of the procedure. On the other hand, the TLR4 response is no longer detectable as early as at the 1^{st} treatment step, which proves that the LPSs have been eliminated. Regarding the NOD2 response, this is suppressed after the ultrafiltration step.

### Conclusion

Although the load of inflammatory compounds has been reduced effectively, procedure 1 is insufficient to ensure complete decontamination of a matrix heavily loaded with PGNs.

Procedure 2: E209J Matrix: Treatment by C EXTRA USP+ENO-PC Activated Carbons; Passage Over Dowex SD2 Resin: 5 kDa Ultrafiltration

The results of the cell tests are given in FIG. 8.

For this procedure, the resin was replaced by Dowex SD2, which has a broader spectrum of retention. The tests were carried out with the E209J matrix, which has a contamination profile similar to that of E1242.

In this case, the response of the Raw-Blue cells is identical to the negative control, which proves that decontamination has been effective.

Nonetheless, the TLR2 response is still higher than the non-contamination control, which indicates that traces of PGN are still present.

The difference in the responses is certainly linked to the fact that the HEK-TLR2 cells have a lower detection threshold for PGNs than the Raw-Blue cells.

The NOD2 response is reduced after passage over SD2 and suppressed after ultrafiltration, which suggests that this resin has an at least complementary action to eliminate the degradation products of PGNs.

### Conclusion

This data indicates that the change in resin has not improved the effectiveness of the procedure in eliminating all the PGNs.

Procedure 3: E5248 Matrix: Treatment by Mannaway® then by C EXTRA USP+a SUPRA EUR Carbons: Passage Over Dowex SD2 Resin: 5 kDa Ultrafiltration

The results of the cell tests are given in FIG. 9.

In this final combination, A SUPRA EUR carbon was combined with C EXTRA USP as replacement for ENO-PC.

Contrary to the latter, A SUPRA EUR carbon preferentially eliminates molecules of high molecular weight.

This combination should be effective for decontaminating samples loaded with aggregated macrocomplexes of PGN or β-glucan type.

The tests were carried out on the E5248 matrix, which induces a low-intensity inflammatory response in the various lines, while assays of SLP-HS and LAL suggest the presence of a strong contamination of PGN and/or f-glucans.

This difference may be explained by the high mass of the inflammatory molecules, which would reduce their solubility, and consequently their accessibility inducing a response in the test cells.

After the 1^{st} step of the procedure, a strong increase in cell responses is observed in the various lines.

This result confirms that the enzymatic treatment by Mannaway® is effective in disaggregating bacterial complexes or debris, thereby releasing agonist molecules for TLR2, TLR4 and NOD2.

After treatment with the carbons, the responses of the Raw cells and the HEK-TLR2 cells have decreased significantly but remain above the negative control.

Likewise, the TLR4 and NOD2 responses remain large, and it is necessary to await the passage over the resin and the final step of ultrafiltration to obtain a signal identical to the non-contamination control in the various cell types.

### Conclusion

These results indicate that A SUPRA EUR carbon has not provided any notable benefit to the decontamination procedure.

### Final Conclusion

Although entirely conceivable, none of these decontamination procedures gives an entirely satisfactory result.

### Example 6: Presentation of the Optimized Procedure

It is chosen to carry out the succession of the following steps on the E3063 matrix.
treatment by Mannaway®, then
by C EXTRA USP+ENO-PC activated carbons, then
passage over Dowex SD2 resin, and finally
5 kDa ultrafiltration.

The results of the cell tests are given in FIG. 10.

To increase the effectiveness of the procedure aiming to eliminate PGNs, a step of enzymatic treatment by Mannaway® is introduced upstream of the other steps.

For these tests, the E3603 matrix, which is very heavily loaded with PGNs, was used.

After the various steps of the procedure, the response of the Raw-Blue cells is identical to the negative control, which proves that decontamination has been effective.

In addition, the combination of enzyme+carbon treatments is particularly suited to eliminating PGNs, since the TLR2 response goes from a saturated signal before treatment to a signal identical to the non-contamination control.

Finally, the other steps are effective in eliminating traces of LPS (TLR4 response) and NOD2 agonists.

In order to demonstrate that this combination is indeed effective, the ENO-PC carbon was removed so as to verify whether its use really provides a benefit to the decontamination procedure.

The results of the cell tests are given in FIG. 11.

The tests were carried out on another matrix, E1565 matrix, which is contaminated with the various agonists of TLR2, TLR4 and NOD2.

At the end of the procedure, the response of the Raw-Blue cells has decreased significantly, but remains slightly greater than the non-contamination control. This very weak response is not linked to the presence of residual PGN but rather to traces of LPS and of NOD2 agonists, and to a possible synergistic effect between the two families of molecules.

The ENO-PC carbon has a broad spectrum of retention for molecules of molecular weight <100 kDa (LPS and degradation products of PGNs).

### Conclusion

It is observed that its absence has reduced the effectiveness of the decontamination procedure, especially if the matrix is heavily contaminated with these two families of inflammatory molecules.

Altogether, the results obtained in this study show that the combination of several decontamination steps carefully selected and placed in order proves effective in eliminating the inflammatory molecules that may be present in the glucose polymer solutions.

The combination comprises the following steps:
treatment by an enzymatic preparation with detergent and clarifying properties, for example Mannaway®,
treatment by an activated carbon of porosity equivalent to C EXTRA USP,
optionally, treatment by a second activated carbon of porosity equivalent to ENO-PC for matrices loaded with degraded PGNs and/or LPS,
passage over an adsorption resin of Dowex-SD2 type,
continuous 5 kDa ultrafiltration.

The steps selected make it possible to target the various families of contaminants and to propose products devoid of inflammatory reactivity.

### FIGURES

FIG. 1: Responses of the Raw-Blue™ cells to standard agonists.

FIG. 2: Responses of the HEK-Blue™ TLR2 cells to standard agonists.

FIG. 3: Responses of the HEK-Blue™ TLR4 cells to standard agonists.

FIG. 4: Responses of the HEK-Blue™ NOD2 cells to standard agonists.

FIG. 5: Responses of the HEK-Blue™ Null cells to standard agonists.

FIG. 6: Cell responses induced by the glucose polymer matrices.

FIG. 7: Cell responses induced by the E1242 matrix after decontamination according to procedure 1.

FIG. 8: Cell responses induced by the E209J matrix after decontamination according to procedure 2.

FIG. 9: Cell responses induced by the E5248 matrix after decontamination according to procedure 5.

FIG. 10: Cell responses induced by the E3063 matrix after decontamination according to procedure 3.

FIG. 11: Cell responses induced by the E1565 matrix after decontamination according to procedure 4.

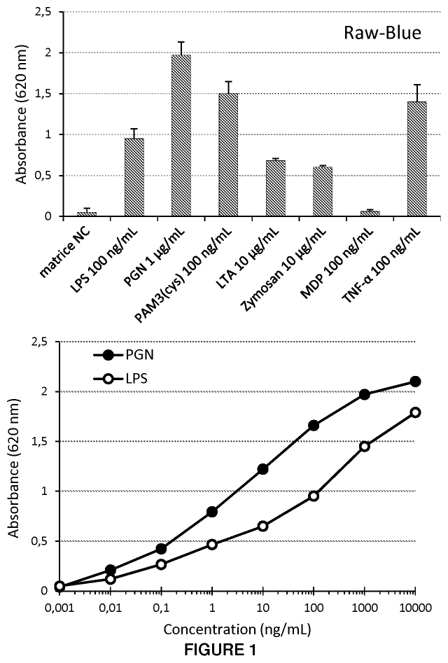

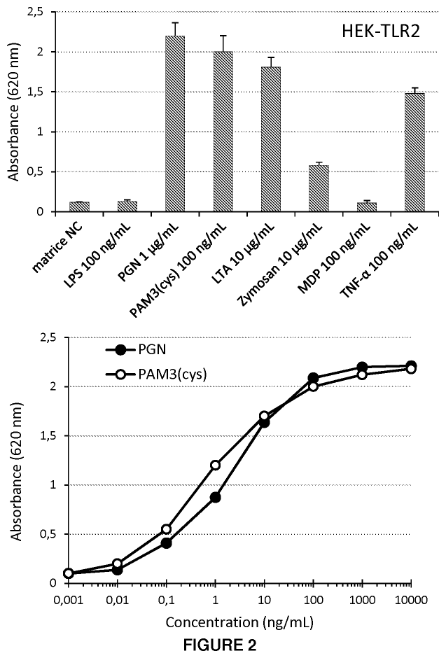

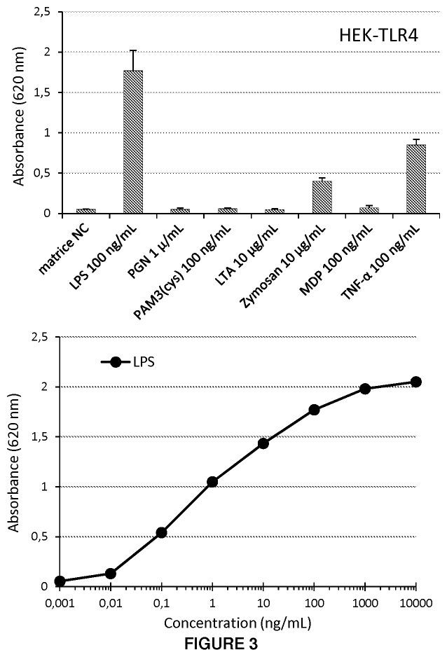

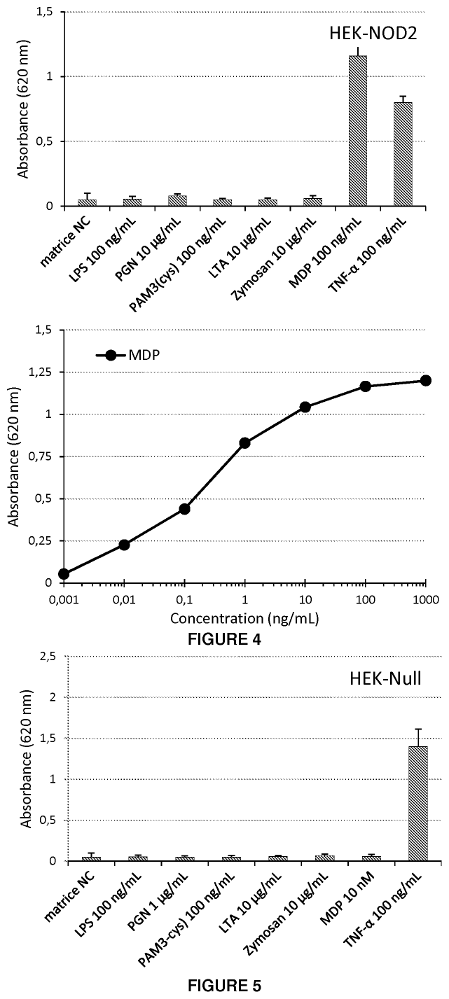

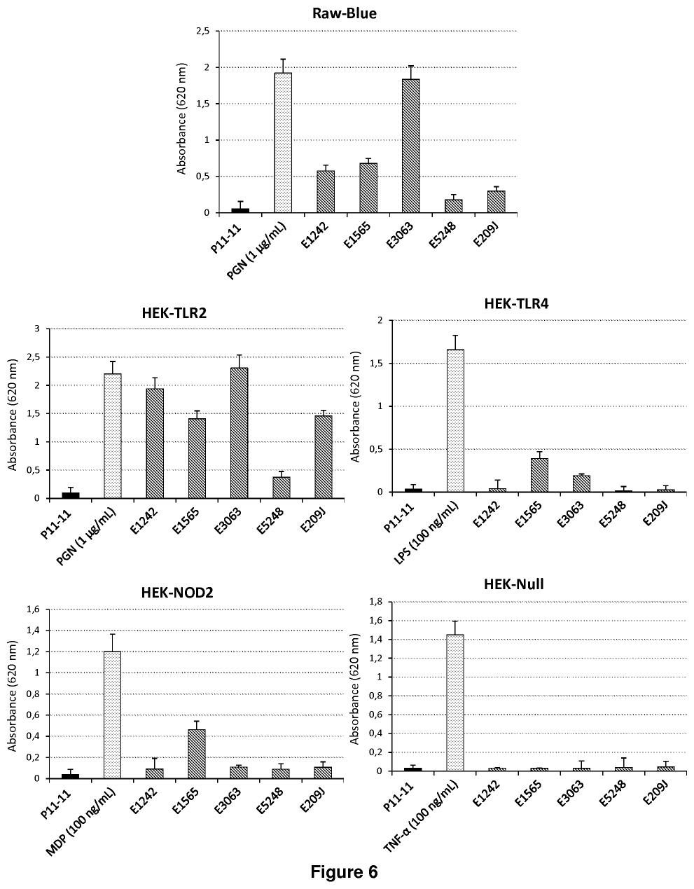

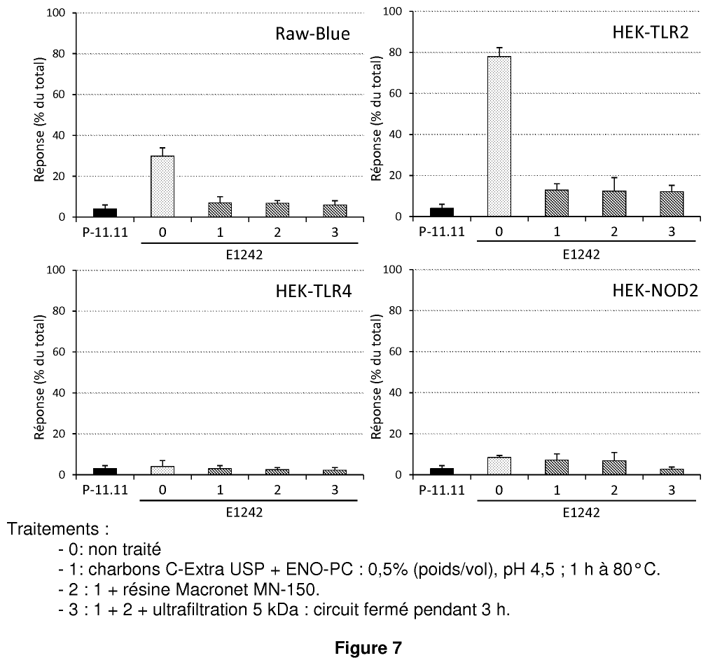

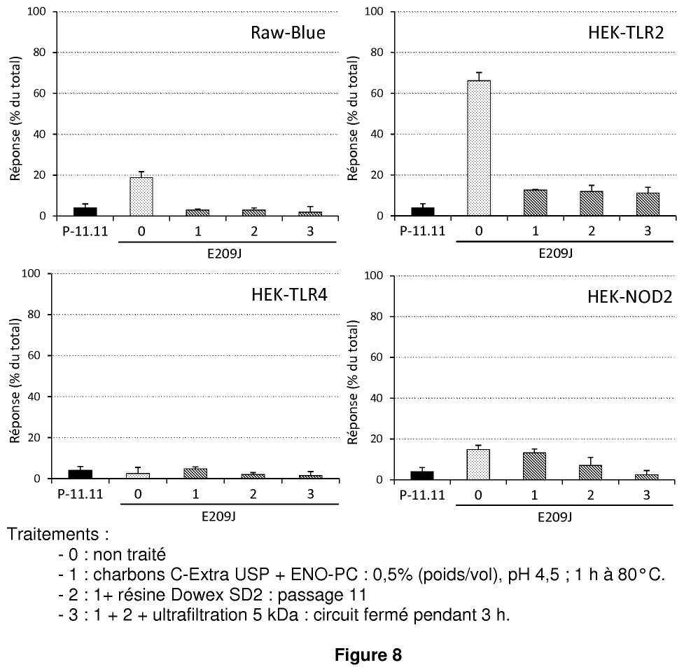

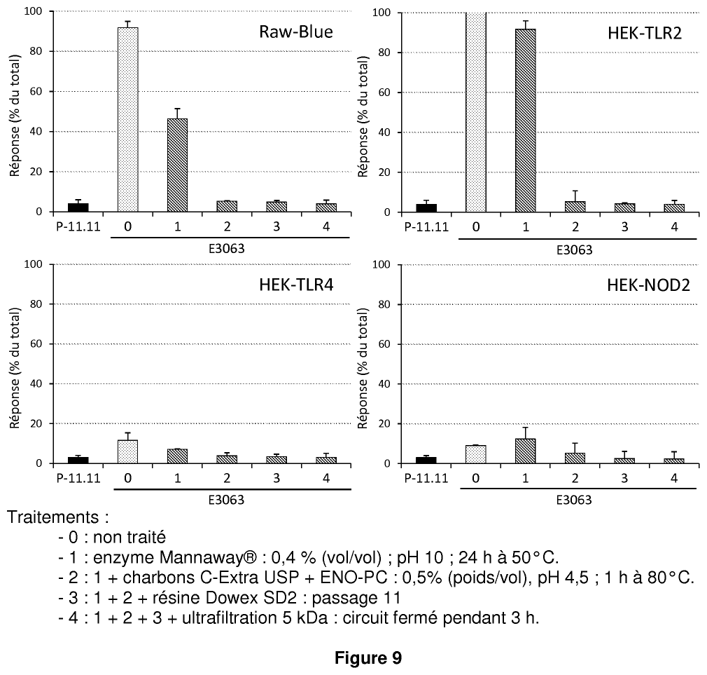

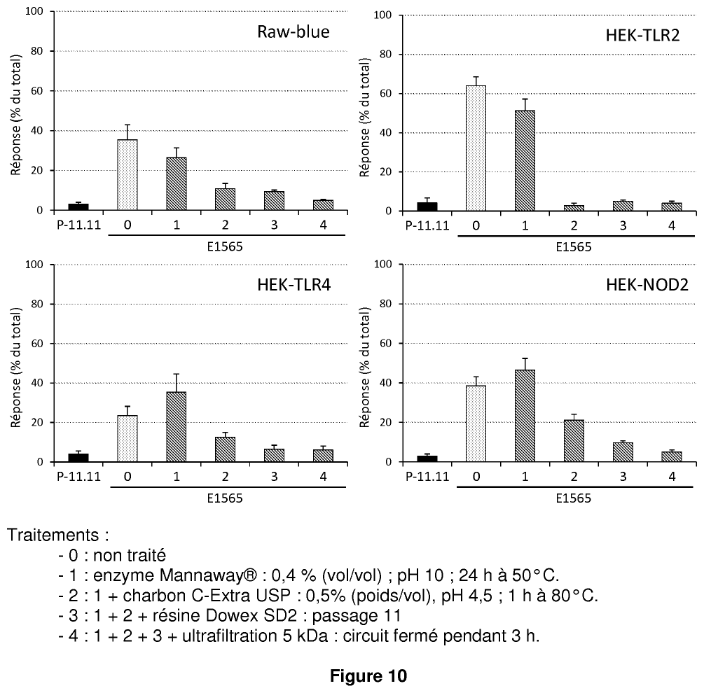

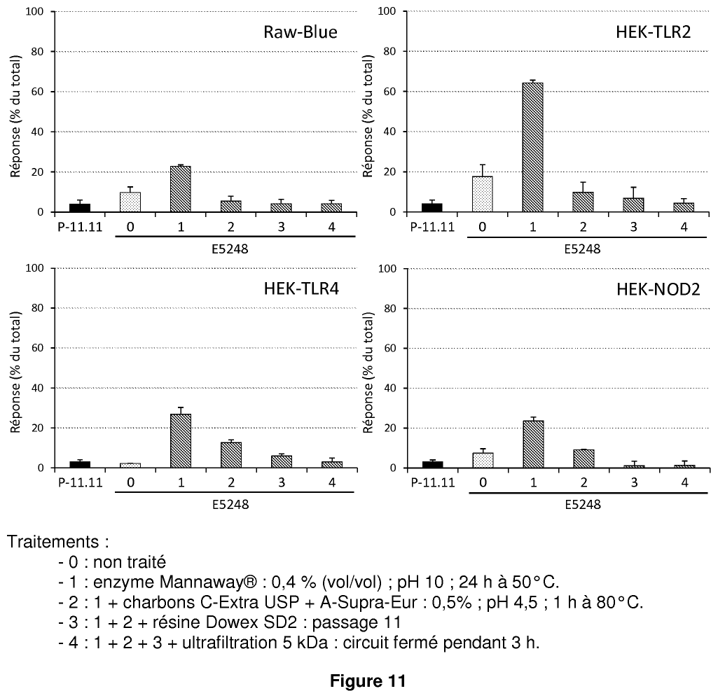

1-7. (canceled)

8. A method for decontaminating glucose polymers or the hydrolyzates thereof of the pro-inflammatory molecules thereof, comprising the following steps:
a) providing glucose polymers or hydrolyzates thereof;
b) optionally, detecting or assaying the pro-inflammatory molecules in the glucose polymers or hydrolyzates thereof provided in step a); and
c) carrying out the following purification steps in the following order:
i) treatment by an enzymatic preparation with detergent and clarifying properties,
ii) treatment by a pharmaceutical-grade activated carbon with very high adsorption capacity and microporous porosity,
iii) treatment by a second activated carbon with mesoporous porosity,
iv) passing over a macroporous adsorbent polymer resin having a pore size greater than 100 angstrom, and
v) continuous 5 kDa ultrafiltration.

9. The method as claimed in claim 8, wherein the enzymatic preparation with detergent and clarifying properties is an enzymatic preparation with mannanase activity.

10. The method as claimed in claim 8, wherein the pharmaceutical-grade activated carbon with very high adsorption capacity and microporous porosity is an activated carbon with porosity equivalent to Norit C Extra USP activated carbon.

11. The method as claimed in claim 8, wherein the activated carbon with mesoporous porosity is an activated carbon with porosity equivalent to ENO-PC activated carbon.

12. The method as claimed in claim 8, wherein the glucose polymers are selected from icodextrin and maltodextrins, and the glucose polymer hydrolyzates are a product of total hydrolysis.

13. The method as claimed in claim 9 wherein the pharmaceutical-grade activated carbon with very high adsorption capacity and microporous porosity is an activated carbon with porosity equivalent to Norit C Extra USP activated carbon.

14. The method as claimed in claim 9, wherein the activated carbon with mesoporous porosity is an activated carbon with porosity equivalent to ENO-PC activated carbon.

15. The method as claimed in claim 9, wherein the glucose polymers are selected from icodextrin and maltodextrins and the glucose polymer hydrolyzates are a product of total hydrolysis.

16. The method as claimed in claim 8, wherein the glucose polymers are selected from branched or unbranched maltodextrins.

17. The method as claimed in claim 8, wherein the glucose polymer hydrolyzates are dextrose monohydrate.

18. The method as claimed in claim 9, wherein the glucose polymers are selected from branched or unbranched maltodextrins.

19. The method as claimed in claim 9, wherein the glucose polymer hydrolyzates are dextrose monohydrate.
